(19) United States
(12) Patent Application Publication
(10) Pub. No.: US 2014/0320807 A1
(43) Pub. Date: Oct. 30, 2014

(54) Method, system and device for providing customized point of care testing

(71) Applicants: Shyam Thangaraju, Chennai (IN); Siva Sakthivel Sadasivam, Chennai (IN)

(72) Inventors: Shyam Thangaraju, Chennai (IN); Siva Sakthivel Sadasivam, Chennai (IN)

(73) Assignee: HCL Technologies Ltd., Chennai (IN)

(21) Appl. No.: 13/874,470

(22) Filed: Apr. 30, 2013

(51) Int. Cl. A61B 5/00; A61B 3/14; A61B 5/145; A61B 10/00; A61B 5/024; A61B 5/053

(52) U.S. Cl. CPC A61B 5/7435; A61B 5/024; A61B 5/0531; A61B 5/145; A61B 10/007; A61B 3/14
USPC 351/206; 600/300; 600/500; 600/547; 600/309; 600/573

(57) ABSTRACT

A method, system and device for providing Point of Care Testing (POCT) are disclosed. The POCT comprises of a device that enables the patient to self conduct plurality of diagnostic tests for a chronic disease such as diabetes mellitus. The method is modular and enables the user to select set of tests from comprehensive list of diagnostic tests. The device provides a strip port to enable testing of various biochemical parameters. Also, the device comprises an ophthalmoscope with a built-in camera to capture images of the retina, a set of piezoelectric sensors for measuring pulse wave velocity (PWV), a pair of electrodes for measuring skin impedance. The method enables the device to log on the test data for future use and analysis. The device can be used for frequent monitoring of health parameters of a user suffering from diabetes mellitus.

The present application claims priority from Indian Application Number 4676/CHE/2012, filed on 7 Nov. 2012, the disclosure of which is hereby incorporated by reference herein.

### TECHNICAL FIELD

The embodiments herein relate to health care systems and more particularly, to Point of Care Testing (POCT) systems.

### BACKGROUND

Health care challenges such as management of chronic illness, reductions of health care costs, improvements in accessibility and quality of health care, provision of care for an aging population are imperative in a society. Point of care testing (POCT) plays a major role in addressing many of the health care challenges. POCT enables diagnostic tests in the immediate vicinity to a patient and provides a rapid result outside the conventional laboratory environment. The quicker test result obtained from POCT permits increased clinical effectiveness by enabling rapid decision making and triage. It also reduces number of outpatient clinic visits.

Chronic illness is widespread and affecting a large section of society. POCT is predominantly used by patients suffering from chronic diseases like diabetes mellitus, who need to frequently monitor their biochemical status. Some existing POCT perform set of tests that include physical and biochemical tests such as detection of fluid flow, determination of pulse rate, determination of heart beat rate, determination of changes in temperature, determination of responses by capillaries, an assessment of flow mediated dilatation, measurements of blood flow and pulse wave velocity, pulse wave analysis, bio-impedance analysis, heart rate variability and measurement of 24 hour blood pressure, however they are not customized for diabetic patients. Such systems with loads of test add complexity in device handling and compel the patient to perform unnecessary tests. Moreover, these systems fail to provide monitoring of eye retina which is critical in detection of vascular damages in a diabetic patient.

Some existing systems are restricted to testing of only eye (retinal) parameters but fail to measure physiological values like skin impedance and pulse wave velocity which is closely correlated to the neurological and vascular health of an individual. Some existing systems provide multiple parameter testing but are not portable, hence fail to seamlessly monitor the patient.

In the light of above discussion, a POCT for measuring multiple health parameters of a patient suffering from diabetic mellitus and customized for diabetic patients will be appreciated.

### SUMMARY

Accordingly the embodiment provides a method for providing customized Point of Care Testing (POCT) for a chronic disease, wherein the method comprises displaying plurality of tests to a user of a device, enabling the user to select at least one test among the plurality of tests, obtaining parameters for the selected test, analyzing the parameters to display test results in the device and storing the results in the device.

Accordingly the embodiment provides a system for providing customized Point of Care Testing (POCT) for a chronic disease, wherein the system comprises a device, at least one sensor, strip, further the system is configured to display plurality of tests to a user of the device, enable the user to select at least one test among the plurality of tests, obtain parameters for the selected test, analyze the parameters to display test results in the device and store the results in the device.

Accordingly the embodiment provides a device providing customized Point of Care Testing (POCT) for a chronic disease, wherein the device comprises, at least one sensor, electrodes, strip port, ophthalmoscope, analog interface, an integrated circuit further comprising at least one processor, at least one memory having a computer program code within the circuit, the at least one memory and the computer program code configured with the at least one processor cause the device to display plurality of tests to a user, enable the user to select at least one test among the plurality of tests, obtain parameters for the selected test, analyze the parameters to display test results and store the results.

### BRIEF DESCRIPTION OF THE FIGURES

The embodiments herein will be better understood from the following detailed description with reference to the drawings, in which:

FIG. 1a, 1b illustrates a device for measurement of health parameters, according to the embodiments disclosed herein;

FIG. 2 illustrates plurality of modules of the device, according to the embodiments disclosed herein; and

FIG. 3 illustrates an exemplary flow diagram explaining testing process of biochemical parameters, according to the embodiments disclosed herein.

### DETAILED DESCRIPTION OF EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The embodiments herein disclose a method, system and device to monitor and analyze health parameters of the patient. The Point of care testing (POCT) disclosed includes a device that enables the patient to self conduct plurality of diagnostic tests. The method is modular and enables the patient to select the set of tests from comprehensive list of tests available. The device provides a strip port to enable testing of biochemical parameters, which have a direct correlation with chronic disease such as diabetes mellitus. The tests results of biochemical parameters indicate the extent of damage to organ systems of patients affected by diabetes mellitus. Further, the device incorporates an ophthalmoscope with a built in camera to capture images of the retina, a set of piezoelectric sensors for measuring pulse wave velocity (PWV) and a pair of electrodes for measuring skin impedance. The method enables the device to log on the test data for future use and analysis. The device can be of great use to diabetic patients who require frequent monitoring of above mentioned health parameters.

The POCT device can be used by patients with diabetes mellitus, physicians in the clinics and laboratories and so on to provide quick test results to enable rapid decision making. These set of diagnostic tests enables early detection of complications at home. It also provides multiple ways to cross verify fundamental features like the vascular and neurologic health of the individual. These tests in combination also improve the reliability and accuracy of the early detection.

In an embodiment, the patient can also be the user of the device. In an embodiment, the device can be a mobile telephone, a cellular phone, a personal communications system (PCS) terminal that may combine a cellular radiotelephone with data processing, facsimile, and/or data communications capabilities, an electronic notepad, a laptop, a personal computer, a tablet, a personal digital assistant (PDA) that can include a telephone, a gaming device or console, a peripheral (e.g., wireless headphone), a digital camera, a media player and the like.

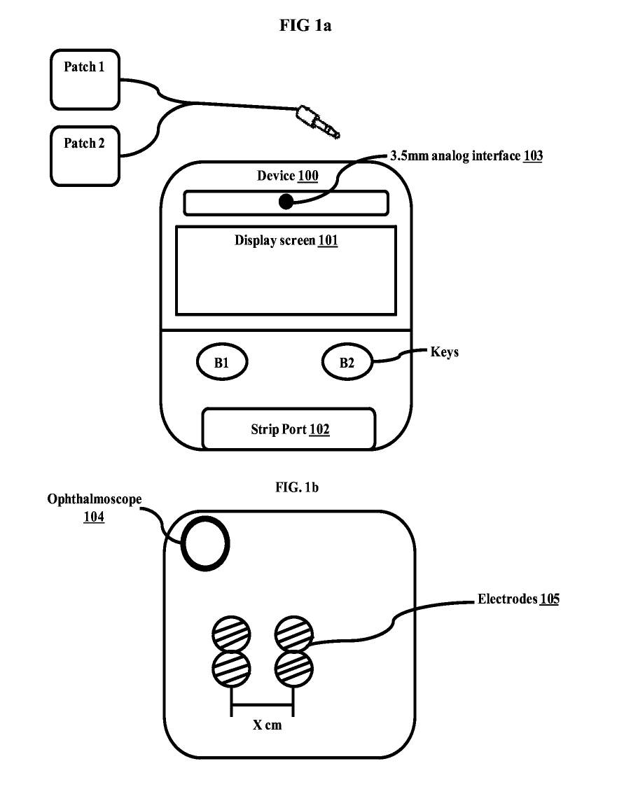

Referring now to the drawings, and more particularly to FIGS. 1a through 3, where similar reference characters denote corresponding features consistently throughout the figures, there are shown embodiments.

FIG. 1a, 1b illustrates a device for measurement of health parameters, according to the embodiments disclosed herein. The FIG. 1a depicts front side of a device 100 comprising of a display screen 101, a strip port 102, a 3.5 mm analog interface 103, keys B1 and B2, patch 1 and patch 2 with a plug-in connector. The FIG. 1b depicts backside of the device 100 comprising of ophthalmoscope 104 and electrodes 105. The device 100 is a POCT device that provides diagnostic tests for chronic diseases such as diabetes mellitus. The device 100 displays information on the display screen 101 that includes list of available tests, selected test, test results and alert messages and so on. The keys B1 and B2 enable the patient to navigate through the displayed menus and perform desired operations such as selecting desired tests, reading previous test records stored in the device 100 and similar functionalities. In an embodiment, the display screen 101 can be a Liquid Crystal Display (LCD), a touch screen, a Light Emitting Diode (LED) backlit LCD screen and so on.

The strip port 102 provides a slot for inserting strip for measurement of biochemical parameters such as parameters of blood, parameters of urine and parameters of saliva and so on.

The analog interface 103 enables connection with an audio jack, further connected to patch 1 and patch 2. The patch 1 and patch 2 are elastic straps equipped with sensors that enable measurement of pulse wave. One of these straps is attached to the upper part of the thigh to detect the pulse wave at the femoral artery and second strap is attached around the ankle to detect the pulse wave of the anterior/posterior tibial artery. These sensors on patch 1 and patch 2 together measure the pulse wave velocity (PWV) through the lower limb. In an embodiment, the sensors can be a pair of piezo electric sensors. The peripheral artery pulse wave velocity is measured by the device 100 and the result displayed on the display screen 101. The PWV of the patients is one of the important factors considered during diagnosis of diabetic patient. The PWV is increased in the initial stages of diabetes mellitus due to increased stiffness of the arterial wall. However, as the disease progresses and as the peripheral arterial disease (PAD) sets in, PWV decreases. Thus, PWV differential between the limbs is an important early indicator of peripheral arterial disease in the limb, and very common in diabetes mellitus.

The device 100 as depicted in FIG. 1b is equipped with an ophthalmoscope 104 that includes a built-in camera. The camera enables the patient to capture the images of the retina and further transmit these images to the physician for analysis. The ophthalmoscope is a lighted instrument that is used to examine the inside of the eye, including the retina and the optic nerve. The retina is one of the earliest regions of the human body where the vascular damage due to diabetes (and hypertension) is immediately apparent. Thus, regular check up of retina by the POCT device 100, provides is the easiest way to identify early damages to the blood vessels in patients with diabetes.

For example, patient uses the ophthalmoscope to capture the retinal image by placing the camera lens near the pupil of the eye. In an embodiment, the camera can be a high performance color CCD camera.

The pair of electrodes 105 enables measurement of the skin impedance of the patient by placing the electrodes 105 over the skin area where the impedance needs to be measured. The skin impedance is significant in diabetic mellitus patients. The skin impedance of persons suffering from peripheral neuropathy increases dramatically. Hence, constant monitoring of skin impedance is vital in order to detect early onset of gangrene in diabetes mellitus patients.

Since all the functionalities are not be required every time or may be unnecessary every time, the device 100 enables the patient to select his/her tests according to his/her needs or as suggested by the physician. For example, while the patient might require measuring blood glucose reading multiple times a day and HbA1C (glycated hemoglobin) can be taken once a month. Liver function tests can be taken once in six months. Similarly, Type-1 diabetes mellitus patients might require more frequent pH (Power of Hydrogen) readings as they are more prone for diabetic keto acidosis. The patients can also monitor the side effects of the drugs. For example, patients taking metformin medication might require frequent testing of pH because lactic acidosis is a side effect of the drug. Thus, the device 100 enables the patients the choice of selecting the tests, from a comprehensive list of tests.

The results of the tests taken by the user are stored in the memory of device 100. The test results are also displayed on the display screen 101 which improves patient understanding of their medical condition and it helps them to take required preliminary measures.

In an embodiment, the device 100 can communicate the results with a PDA, smart phone, tablet, laptop, a personal computer which is programmed to encourage diet and exercise.

In an embodiment, the test result data can be logged on to a remote server and can later be accessed by any authorized person for further diagnosis. Integration of the test results data provided by the device 100 to the patient's case sheet improves the physician's understanding of the patient's profile.

For example, the output of the POCT device 100 is made available immediately within an electronic medical record. Further, this medical record (test results) can be shared instantaneously with all members of the medical team through communication network. This helps to reduce the turnaround time during diagnosis.

POCT improves the motivation level of patients to maintain their health and improves their compliance for medication. POCT can also be used as pay per use model.

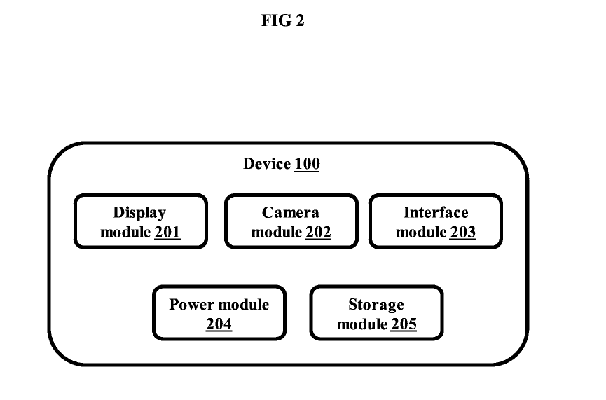

FIG. 2 illustrates plurality of modules of the device, according to the embodiments disclosed herein. The figure depicts the device 100 that comprises a display module 201, a camera module 202, an interface module 203, a power module 204 and a storage module 205. The display module 201 provides the user interface (UI) to the user to customize the tests and to navigate. The display module 201 displays the menu that includes comprehensive list of all diagnostic tests. The display module 201 displays results of the tests on the display screen 101, providing a visual representation of the test data. The camera module 202 comprises an ophthalmoscope with built-in camera.

The interface module 203 provides input and/or output interface for data communication. In an embodiment, the interface module 203 receives measured parametric data from piezo electric sensors, pair of electrodes, strip port and so on. For example, the input data is from piezo electric sensors measuring the PWV.

In other example, the input data is the biochemical parameter at the strip port 102. The interface module 203 also provides communication interface that enables the device 100 to communicate with external devices to transmit the result data.

In an embodiment, the communication can be a wired communication, wireless communication. For example, wired communication includes but not limited to USB, UART, and Ethernet. For example, wireless communication includes but not limited to cellular network, GPRS, Wi-Fi, and Bluetooth. The communication interface provided by the interface module 203 enables the device 100 to log on the test result data on to the server and thus provides better test result data accessibility (anywhere anytime) by any authorized person.

The power module 204 includes a battery to power up the device 100. The power module 204 includes a battery recharging unit that can be connected to an adapter or a USB charger. The storage module 205 includes a memory to store the test result data. In an embodiment, the memory can be a Read Only Memory (ROM), flash memory and so on.

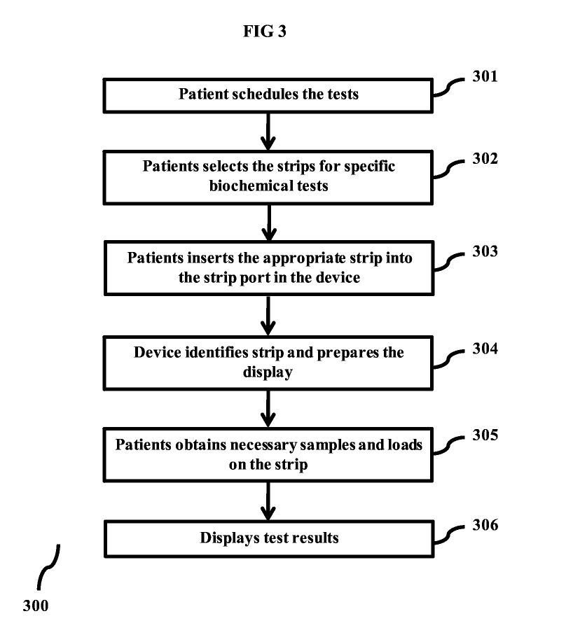

FIG. 3 illustrates an exemplary flow diagram explaining testing process of biochemical parameters, according to the embodiments disclosed herein. As depicted in flow diagram 300 the patient schedules (301) the required tests and decide the date and time of the test required. In an embodiment, the patient may receive test schedule from the physician or can self schedule the test as required. Then the patient activates the device 100. Further, from the comprehensive list of available tests displayed on the display screen 101, patient selects the biochemical tests. The patient uses various standard reagent test strips for testing of various parameters of blood, urine, saliva and so on. The patient selects (302) the required strip for biochemical parameter to be measured and inserts (303) the appropriate strip into the strip port 102 in the device 100. The device 100 identifies (304) the strip and the display module 201 customizes the display for identified test. The patient obtains (305) either of the blood, urine or saliva samples and loads them in the slot provided on the strip. The device 100 analyzes the strips and displays (306) the test result readings on the display screen 101. The device 100 stores the test results for future use. The various actions in flow diagram 300 may be performed in the order presented, in a different order or simultaneously. Further, in some embodiments, some actions listed in FIG. 3 may be omitted.

In an embodiment, device 100 measures blood parameters such as glucose, HbA1C(glycated haemoglobin), pH (Power of Hydrogen), conjugated and un-conjugated bilirubin, Serum Glutamic Oxaloacetic Transaminase (SGOT) and Serum Glutamic Pyruvic Transaminase (SGPT), albumin, globulin, lipid profile, blood urea and creatinine and so on.

In an embodiment, device 100 measures urine parameters such as pH, glucose, protein levels, and ketone bodies, urea and creatinine and so on.

The embodiments disclosed herein can be implemented through at least one software program running on at least one hardware device and performing network management functions to control the network elements. The network elements shown in FIGS. 1 and 2 include blocks which can be at least one of a hardware device, or a combination of hardware device and software module.

The embodiment disclosed herein specifies a system for providing customized Point of Care Testing (POCT) for a chronic disease. The mechanism allows customization of POCT by providing a system thereof. Therefore, it is understood that the scope of the protection is extended to such a program and in addition to a computer readable means having a message therein, such computer readable storage means contain program code means for implementation of one or more steps of the method, when the program runs on a server or mobile device or any suitable programmable device.

The method is implemented in a preferred embodiment through or together with a software program written in e.g. Very high speed integrated circuit Hardware Description Language (VHDL) another programming language, or implemented by one or more VHDL or several software modules being executed on at least one hardware device. The hardware device can be any kind of device which can be programmed including e.g. any kind of computer like a server or a personal computer, or the like, or any combination thereof, e.g. one processor and two FPGAs. The device may also include means which could be e.g. hardware means like e.g. an ASIC, or a combination of hardware and software means, e.g. an ASIC and an FPGA, or at least one microprocessor and at least one memory with software modules located therein. Thus, the means are at least one hardware means and/or at least one software means. The method embodiments described herein could be implemented in pure hardware or partly in hardware and partly in software. The device may also include only software means. Alternatively, the embodiment may be implemented on different hardware devices, e.g. using a plurality of CPUs.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the claims as described herein.

What is claimed is:

1. A method for providing customized Point of Care Testing (POCT) for a chronic disease, wherein said method comprises:
displaying plurality of tests to a user of a device;
enabling said user to select at least one test among said plurality of tests;
obtaining parameters for said selected test;
analyzing said parameters to display test results in said device; and
storing said results in said device.

2. The method as in claim 1, wherein said chronic disease is a diabetes mellitus.

3. The method as in claim 1, wherein said parameters comprises at least one of: biochemical parameters, Pulse Wave Velocity (PWV), skin impedance, eye parameters, wherein said biochemical parameters are measured for at least one of: blood, urine, saliva.

4. The method as in claim 3, wherein said biochemical parameters are measured using a strip.

5. A system for providing customized Point of Care Testing (POCT) for a chronic disease, wherein said system comprises a device, at least one sensor, strip, further said system is configured to:
display plurality of tests to a user of said device;
enable said user to select at least one test among said plurality of tests;
obtain parameters for said selected test;
analyze said parameters to display test results in said device; and
store said results in said device.

6. The system as in claim 5, wherein said system is configured to display said plurality of tests using a display module in said device.

7. The system as in claim 5, wherein said parameters comprises at least one of: biochemical parameters, Pulse Wave Velocity (PWV), skin impedance, eye parameters, wherein said biochemical parameters are measured for at least one of: blood, urine, saliva.

8. The system as in claim 7, wherein said system is configured to measure said biochemical parameters of at least one of: blood, urine, saliva using said strip.

9. The system as in claim 7, wherein said system is configured to measure said Pulse Wave Velocity (PWV) using said at least one sensor and measure said skin impedance using said electrodes.

10. The system as in claim 7, wherein said eye parameters are measured using an ophthalmoscope with built-in camera module integrated in said device.

11. The system as in claim 5, wherein said system is configured to store said results in a storage module in said device.

12. A device providing customized Point of Care Testing (POCT) for a chronic disease, wherein said device comprises:
at least one sensor,
electrodes,
strip port,
ophthalmoscope,
analog interface;
an integrated circuit further comprising at least one processor;
at least one memory having a computer program code within said circuit;
said at least one memory and said computer program code configured to with said at least one processor cause said device to:
display plurality of tests to a user;
enable said user to select at least one test among said plurality of tests;
obtain parameters for said selected test;
analyze said parameters to display test results; and
store said results.

13. The device as in claim 12, wherein said chronic disease is a diabetes mellitus.

14. The device as in claim 12, wherein said device is configured to measure said parameters, wherein said parameters comprises at least one of: biochemical parameters, Pulse Wave Velocity (PWV), skin impedance, eye parameters, wherein said biochemical parameters are measured for at least one of: blood, urine, saliva.

15. The device as in claim 14, wherein said device is configured to measure said Pulse Wave Velocity (PWV) using said at least one sensor connected with said analog interface and measure said skin impedance using said electrodes.

16. The device as in claim 14, wherein said device is configured to measure said eye parameters using said ophthalmoscope with built-in camera module.

17. The device as in claim 12, wherein said device is configured to communicate said stored results to an external device, wherein said communication comprises at least one of: wired, wireless.
